SEQUENCE LISTING (3 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 19 aa, synthetic construct
MQIELSTCFFLCLLRFCFS

SEQ ID NO: 3 - protein, 14 aa, synthetic construct
SFSQNPPVLKRHQR

(19) United States
(12) Patent Application Publication
(10) Pub. No.: US 2026/0242823 A1
(43) Pub. Date: Aug. 20, 2026

(54) COMPOSITIONS AND METHODS FOR MODULATING FACTOR VIII FUNCTION

(71) Applicant: The Children's Hospital of Philadelphia, Philadelphia, PA (US)

(72) Inventors: Lindsey A. George, Haddonfield, NJ (US); Rodney M. Camire, Sicklerville, NJ (US)

(21) Appl. No.: 19/129,482

(22) PCT Filed: Dec. 11, 2023
(86) PCT No.: PCT/US2023/083309

§ 371 Date: May 13, 2025

Related U.S. Application Data
(60) Provisional application No. 63/431,461, filed on Dec. 9, 2022.

(51) Int. Cl. C12N 15/86; A61K 35/76; A61K 38/00; A61K 48/00; A61P 7/04; C07K 14/755

(52) U.S. Cl. CPC C12N 15/86; A61K 35/76; A61K 48/0008; A61K 48/005; A61P 7/04; C07K 14/755; A61K 38/00; C12N 2750/14143

(57) ABSTRACT

Factor VIII variants and methods of use thereof are disclosed.

This application claims priority under 35 U.S.C. § 119(e) to U.S. Provisional Patent Application No. 63/431,461, filed Dec. 9, 2022. The foregoing application is incorporated by reference herein.

This invention was made with government support under Grant Number NHLBI HL146991 awarded by the National Institutes of Health. The government has certain rights in the invention.

### FIELD OF THE INVENTION

The present invention relates to the fields of medicine and hematology. More specifically, the invention provides novel Factor VIII variants and methods of using the same to modulate the coagulation cascade in patients in need thereof.

### BACKGROUND OF THE INVENTION

Several publications and patent documents are cited throughout the specification in order to describe the state of the art to which this invention pertains. Each of these citations is incorporated herein by reference as though set forth in full.

Coagulation factor VIII (FVIII) circulates in blood tightly bound to its carrier protein, von Willebrand factor (vWF) (Eaton, et al. (1986) Biochemistry 25(2):505-512; Vehar, et al. (1984) Nature 312(5992):337-342; Lollar, et al. (1988) J. Biol. Chem., 263(21):10451-10455). Proteolytic processing by thrombin liberates FVIII from vWF and produces the active cofactor species (FVIIIa), which is a heterotrimer comprised of an A2-domain weakly associated with the metal ion-stabilized A1/A3-C1-C2 heterodimer (Vehar, et al. (1984) Nature (1984) 312(5992):337-342; Fay, et al. (1992) J. Biol. Chem., 267(19):13246-13250). Factor VIIIa associates with activated FIX (FIXa) on anionic phospholipid surfaces forming the intrinsic Xase enzyme complex, one of two enzymes that activates FX (Eaton, et al. (1986) Biochemistry 25(2):505-512; Hill-Eubanks, et al. (1990) J. Biol. Chem., 265(29):17854-17858; Lenting, et al. (1994) J. Biol. Chem., 269(10):7150-7155; Venkateswarlu, D. (2014) Biochem. Biophys. Res. Comm., 452(3):408-414; Kolkman, et al. (1999) Biochem J., 339(Pt 2):217-221; Fay, et al. (1998) J. Biol. Chem., 273(30):19049-19054; Kolkman, et al. (1999) 274(41):29087-29093; Kolkman, et al. (2000) Biochemistry 39(25):7398-7405). Deficiency or dysfunction of FVIII results in hemophilia A (HA), highlighting the importance of FVIIIa cofactor function. Downregulation of intrinsic Xase function is achieved through inhibition of FIXa by antithrombin and possibly protein S (PS), and FVIIIa inactivation by spontaneous A2-domain dissociation or proteolytic cleavage at Arg336 and Arg562 by activated protein C (APC) (Lollar, et al. (1991) J. Biol. Chem., 266(19):12481-12486; Hultin, et al. (1981) Blood 57(3):476-482; Lollar, et al. (1984) Blood 63(6):1303-1308; Lollar, et al. (1990) J. Biol. Chem., 265(3):1688-1692; Walker, et al. (1987) Arch. Biochem. Biophys., 252(1):322-328; Plautz, et al. (2018) Arterioscler. Thromb. Vasc. Biol., 38(4):816-828; Fay, et al. (1991) J. Biol. Chem., 266(30):20139-20145). Because FVIIIa has such a profound effect (10³-10⁶-fold) on increasing FIXa function, its inactivation is important for regulating intrinsic Xase function (van Dieijen, et al. (1981) J. Biol. Chem., 256(7):3433-3442; Mertens, et al. (1984) Biochem. J., 223(3):599-605).

Mutations in Factor VIII (FVIII) can lead to severe bleeding disorders and are associated with hemophilia A. Defective FVIII or a lack of FVIII activity results in an inability to effectively form clots. To date, only 20% of patients with hemophilia A worldwide receive regular treatment with FVIII replacement therapy due its high cost. Typically, FVIII therapy is plasma-derived or recombinantly produced. Gene therapy for hemophilia A based on AAV vectors is promising. Generating enhanced function FVIII variants would benefit the treatment of hemophilia. Therefore, there is an obvious need for FVIII molecules with improved biological properties.

### SUMMARY OF THE INVENTION

In accordance with the present invention, compositions and methods for the modulation of hemostasis in patients in need thereof are provided. More specifically, Factor VIII (FVIII) variants which modulate (e.g., increase) hemostasis or blood coagulation are provided. In certain embodiments, the Factor VIII variant is more resistant to cleavage by a serine protease than wild-type. In certain embodiments, the Factor VIII variant comprises at least one mutation at position 336, 562, 372, 740, and/or 1689. In certain embodiments, the Factor VIII variant comprises a mutation at positions 336 and 562 and one, two, or three mutations at positions 372, 740, or 1689. In certain embodiments, the Arg at position 336, 562, 372, 740, and/or 1689 is substituted with Gln. Compositions comprising at least one FVIII variant of the instant invention and at least one pharmaceutically acceptable carrier are also provided. Nucleic acid molecules encoding the FVIII variants of the invention, including vectors such as viral vectors, are also disclosed as are methods of use thereof. Another aspect of the invention includes host cells expressing the FVIII variants described herein. Methods for isolating and purifying the FVIII variants are also disclosed.

Pharmaceutical compositions comprising the FVIII variants and/or FVIII variant encoding nucleic acid molecules of the invention in a carrier are also provided. The invention also includes gene therapy methods. The invention also includes methods for the treatment of a hemostasis related disorder in a patient in need thereof or for the promotion or increase of blood coagulation in a patient in need thereof comprising administration of a therapeutically effective amount of the FVIII variant and/or FVIII variant encoding nucleic acid molecules, particularly within a pharmaceutical composition. The instant invention also encompasses methods of increasing FVIII expression (e.g., in vivo), increasing plasma concentration of FVIII, and/or increasing steady-state levels of FVIII. Such methods have efficacy in the treatment of disorders where a pro-coagulant is needed and include, without limitation, hemophilia, particularly hemophilia A.

### BRIEF DESCRIPTION OF THE DRAWINGS

FIG. 1A provides an amino acid sequence of FVIII (SEQ ID NO: 1). The amino acids at positions 336 and 562 are bolded and underlined. The B domain is also indicated with italics and bolding. The thrombin cleavage site arginines at 372, 740, and 1689 are indicated by italics and underlining. The provided amino acid sequence lacks the 19 amino acid signal peptide at the N-terminus (MQIELSTCFFLCLLRFCFS (SEQ ID NO: 2)). FIG. 1B provides a schematic of the FVIII domain structure with thrombin (IIa) and APC cleavage sites noted.

FIG. 2 provides a graph of FVIII plasma concentrations in mice treated with 5×10¹¹ or 1×10¹² vector genomes (vg)/mouse of an adeno-associated virus (AAV) vector which expressed FVIII-QQ or wild-type FVIII (FVIII-WT). 1 nM is approximately normal plasma FVIII. n=6-8 mice/cohort. Two-way ANOVA with Sidak's multiple comparisons test: ** p<0.01, *** p<0.001.

FIG. 3A provides a schematic of CRISPR/Cas9 generation of a FVIII-QQ mouse model. Mice are naturally R336Q. Accordingly, only the R562Q mutation was introduced. FIG. 3B provides a graph of plasma FVIII activity in FVIII-WT mice and FVIII-QQ mice.

FIG. 4 provides a graph of plasma FVIII-QQ and FVIII-WT expression following AAV vector administration. Each data point represents a cohort of 6-11 mice treated with the same vector and dose, differing only by the 2 amino acids of FVIII-QQ compared to FVIII-WT. Data are plotted on a log scale. The dotted line represents a 1:1 correlation. The dashed horizontal line denotes 0.5 nM. Data represent approximately a total of 130 AAV treated mice. All data are from mice in which the vector was known to have equal FVIII-QQ and FVIII-WT mRNA and intracellular FVIII from harvested hepatocytes.

FIG. 5A provides a graph of blood loss from a tail clip assay for HA/CD4KO mice treated with an equal AAV vector dose and construct to express FVIII-WT or FVIII-QQ. FIG. 5B provides graphs of FVIII (F8) mRNA, intracellular FVIII, and FVIII plasma concentration in HA/CD4KO mice treated with an equal AAV vector dose and construct to express FVIII-WT (squares) or FVIII-QQ (triangles). Similar data from the CRISPR/Cas9 FVIII-QQ mouse model compared to wild-type littermate controls. Unpaired t-test: ** p<0.01, **** p<0.0001.

FIG. 6 provides graphs of FVIII (F8) mRNA, intracellular FVIII, and media FVIII concentration in BHK stable cells expressing FVIII-WT (squares) or FVIII-QQ (triangles).

FIG. 7 provides Western blot images of FVIII-WT or FVIII-QQ and the indicated serine proteases. Cleavage fragments at R336 and/or R562 are present in FVIII-WT but not FVIII-QQ. Cleavage at R372 is present in both FVIII-WT and FVIII-QQ for all tested serine proteases.

FIG. 8 provides Western blot images of FVIII-WT or FVIII-QQ in the presence of vWF and the indicated serine proteases. Cleavage fragments at R336 and/or R562 are present in FVIII-WT but not FVIII-QQ. Cleavage at R372 is present in both FVIII-WT and FVIII-QQ for all tested serine proteases.

FIG. 9 provides Western blot images of FVIII-WT, FVIII-QQ, and FVIIIR372Q in the presence of vWF and the indicated serine proteases. Cleavage fragments at R336 and/or R562 are present in FVIII-WT but not FVIII-QQ. Cleavage at R372 is present in both FVIII-WT and FVIII-QQ for all tested serine proteases, but not in the FVIII-R372Q control.

FIG. 10 provides a graph of survival studies in HA/CD4KO and WT/CD4KO mice treated with AAV vectors to express human FVIII-WT and FVIII-QQ in the range of mild HA, normal, and supratherapeutic FVIII levels.

### DETAILED DESCRIPTION OF THE INVENTION

Hemophilia A (HA) and hemophilia B (HB) are X-linked bleeding disorders due to inheritable deficiencies in either coagulation factor VIII (FVIII) or factor IX (FIX), respectively (Peyvandi, et al., Lancet (2016) 388:187-197; Konkle, et al., Hemophilia A in GeneReviews, Adam, et al., eds., University of Washington (1993)). The bleeding phenotype is generally related to the residual factor activity: people with severe disease (factor activity <1% normal) have frequent spontaneous bleeds; people with moderate disease (factor activity 1%-5% normal) rarely have spontaneous bleeds, but bleed with minor trauma; and people with mild disease (factor activity 5%-40% normal) bleed during invasive procedures or trauma. Given this well-defined relationship between factor activity and bleeding phenotype, HA and HB are attractive targets for protein infusion or gene therapy as small increases in factor levels are expected to have a meaningful clinical impact.

As explained above, Factor VIII is central for coagulation activity and mutations in the FVIII gene result in hemophilia A, the most common form of hemophilia. Herein, specific changes in the amino acid sequence of FVIII are shown to be associated with enhanced protein resistance to proteolytic inactivation. Thus, the instant invention provides rationally designed amino acid residue modifications which provide unexpectedly superior variants.

Full-length FVIII is a large, 280-kDa protein primarily expressed in liver sinusoidal endothelial cells (LSECs), as well as extra-hepatic endothelial cells (Fahs, et al., Blood (2014) 123:3706-3713; Everett, et al., Blood (2014) 123:3697-3705). FVIII predominantly circulates as a heterodimer of a heavy chain and a light chain bound through noncovalent metal-dependent interactions (Lenting, et al., Blood (1998) 92:3983-3996). Factor VIII comprises several domains and is 2332 amino acids in length (mature without signal peptide). Generally, the domains are referred to as A1-A2-B-A3-C1-C2. FVIII is translated as a single-peptide chain (single chain) with the domain structure of A1-α1-A2-α2-B-α3-A3-C1-C2. Proteolytic cleavage of FVIII at R-1313 and/or R-1648 by the trans-Golgi protease furin results in heterodimer formation. The FVIII heavy chain (A1-α1-A2-α2-B) and light chain (α3-A3-C1-C2) remain associated through non-covalent metal-ion-dependent interactions occurring between the A1 and A3 domains. Initially, FVIII is in an inactive form bound to von Willebrand factor (vWF). FVIII is activated by cleavage by thrombin (Factor IIa) and release of the B domain. The activated form of FVIII (FVIIIa) separates from vWF and interacts with coagulation factor Factor IXa—leading to the formation of a blood clot via a coagulation cascade. During coagulation, FVIII single chain or heterodimer is activated to its heterotrimeric cofactor form by cleavage by thrombin at R-372, R-740, and R-1689. A2 remains associated with A1-α1 via non-covalent interactions. Inactivation of FVIIIa occurs via spontaneous A2 dissociation and/or proteolytic cleavage, primarily by activated protein C, at R-336 and R-562. FVIIIa is a cofactor for FIXa within the intrinsic Xase complex which functions to generate FXa, leading to the propagation of the coagulation cascade.

The B domain comprises 40% of the protein (908 amino acids) and is not required for the protein procoagulant activity (Brinkhous, et al., Proc. Natl. Acad. Sci. (1985) 82:8752-8756). The most common B-domain deleted (BDD) FVIII comprises 14 original amino acid residues (SFSQNPPVLKRHQR (SEQ ID NO: 3)) as a linker (Lind, et al. (1995) Eur. J. Biochem., 232(1):19-27). This BDD FVIII is typically referred to as BDD-SQ or hFVIII-SQ. Short peptide linkers (e.g., 25 or fewer amino acids, 20 or fewer amino acids, 15 or fewer amino acids, or 10 or fewer amino acids—optionally derived directly from the B domain (e.g., beginning at the amino terminus of the B domain)) substituted for the B-domain can be used in FVIII variants (Lind, et al. (1995) Eur. J. Biochem., 232(1):19-27; Pittman, et al., Blood (1993) 81:2925-2935; Toole, et al., Proc. Natl. Acad. Sci. (1986) 83:5939-5942). In a particular embodiment, the peptide linker comprises a basic amino acid (e.g., Arg, His, or Lys) at position −1 and −4 to Glu1649. This BDD FVIII form is commonly used to produce recombinant BDD-FVIII (~4.4 Kb) as well for gene therapy (Berntorp, E., Semin. Hematol. (2001) 38(2 Suppl 4):1-3; Gouw, et al., N. Engl. J. Med. (2013) 368:231-239; Xi, et al., J. Thromb. Haemost. (2013) 11:1655-1662; Recht, et al., Haemophilia (2009) 15:869-880; Sabatino, et al., Mol. Ther. (2011) 19:442-449; Scallan, et al., Blood (2003) 102:2031-2037). As noted above, gene therapy using AAV vectors can only use shortened FVIII molecules such as a BDD-FVIII due to the limited packaging capacity of the AAV (4.7 Kb) and other vector systems (Lind, et al. (1995) Eur. J. Biochem., 232(1):19-27). U.S. Pat. No. 8,816,054, incorporated by reference herein, also provides BDD FVIII molecules with linkers of different lengths and sequences.

In accordance with the instant invention, novel Factor VIII variants are provided. The instant invention encompasses FVIII variants including FVIIIa variants and FVIII prepeptide variants. For simplicity, the variants are generally described throughout the application in the context of FVIII. However, the invention contemplates and encompasses Factor FVIIIa and FVIII prepeptide molecules as well as Factor VIII domain(s) (e.g., A1 and/or A2 domain) with amino acid substitutions as described. In a particular embodiment, the FVIII variants are B-domain deleted (BDD) FVIII (optionally comprising a linker in place of the B-domain). In a particular embodiment, the FVIII variants comprise A1-α1-A2-α2-B-α3-A3-C1-C2. In a particular embodiment, the FVIII variants comprise A1-α1-A2-α2-α3-A3-C1-C2. In a particular embodiment, the FVIII variants comprise A1-α1-A2-α2-A3-C1-C2. In a particular embodiment, the FVIII variants comprise a light chain and a heavy chain.

As demonstrated herein, the FVIII variants of the instant invention unexpectedly possess greater expression, particularly in vivo, and increased plasma concentrations and steady-state levels compared to, e.g., wild-type FVIII.

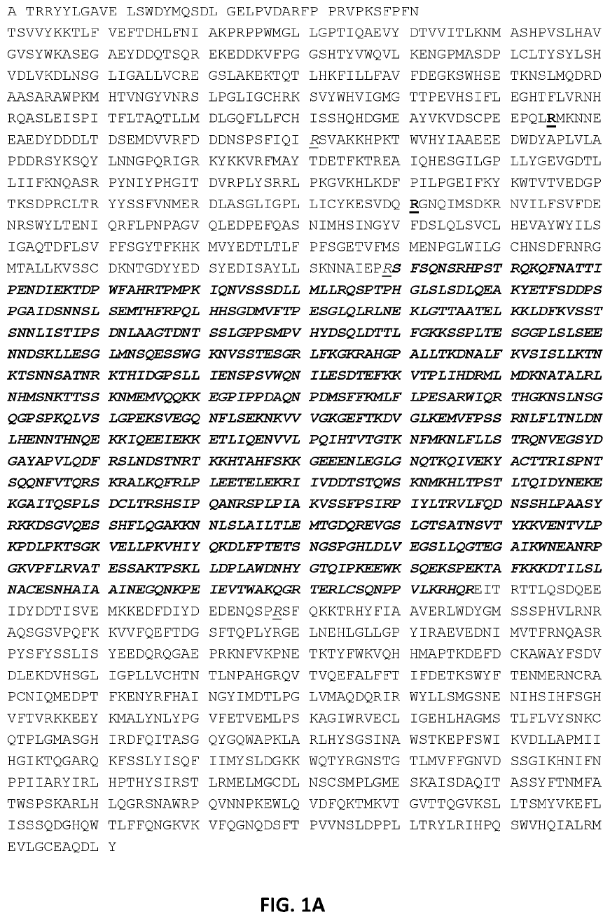

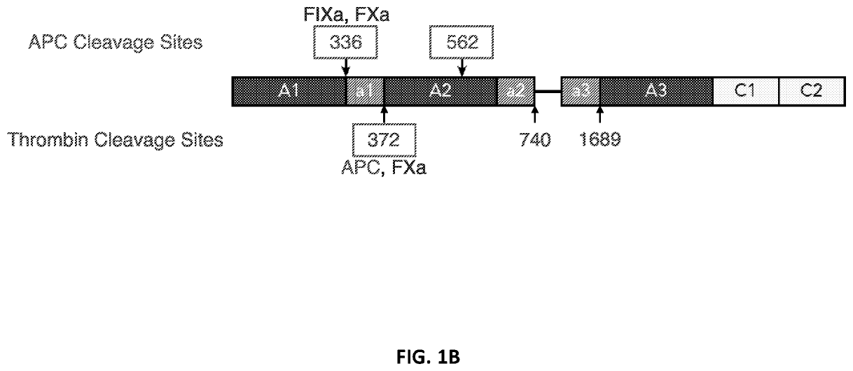

The FVIII variants of the instant invention can be from any mammalian species. In a particular embodiment, the FVIII variant is human. Gene ID: 2157 and GenBank Accession Nos. NM_000132.3 and NP_000123.1 provide examples of the amino acid and nucleotide sequences of wild-type human FVIII (particularly the prepeptide comprising the signal peptide). FIG. 1 provides SEQ ID NO: 1, which is an example of the amino acid sequence of human FVIII. SEQ ID NO: 1 lacks the 19 amino acid signal peptide at its N-terminus (MQIELSTCFFLCLLRFCFS (SEQ ID NO: 2)), although the FVIII variants of the instant invention may comprise the signal peptide. Nucleic acid molecules which encode Factor FVIII variants can be readily determined from the provided amino acid sequences as well as the provided GenBank Accession Nos.

The Factor VIII variants of the instant invention may comprise a mutation(s) which provides resistance to cleavage by a serine protease. For example, the cleavage site for the serine protease may be mutated with one or more substitutions. In certain embodiments, the Factor VIII variants of the instant invention may be cleaved at less than 50%, at less than 40%, at less than 30%, at less than 20%, at less than 10%, at less than 5%, or at less than 1% of the cleavage of wild-type FVIII (e.g., in an in vitro assay with the serine protease).

In certain embodiments, the serine protease is selected from the group consisting of APC, FIXa, FXa, thrombin (IIa), and plasmin. The Factor VIII variants of the instant invention may comprise at least one mutation at position 336, 562, 372, 740, and/or 1689. In certain embodiments, the Factor VIII variant comprises at least two mutations at positions 336, 562, 372, 740, and/or 1689. In certain embodiments, the Factor VIII variant comprises at least three mutations at positions 336, 562, 372, 740, and/or 1689. In certain embodiments, the Factor VIII variant comprises at least four mutations at positions 336, 562, 372, 740, and/or 1689. In certain embodiments, the Factor VIII variant comprises a mutation at positions 336, 562, 372, 740, and 1689. In certain embodiments, the Factor VIII variant comprises a mutation at positions 336 and 562 and, optionally, one, two, or three mutations at positions 372, 740, and/or 1689. In certain embodiments, the Factor VIII variant comprises R336Q and R562Q and one, two, or three mutations at positions 372, 740, and/or 1689.

In certain embodiments, the Factor VIII variants comprise a mutation at position 336. In a particular embodiment, the Arg (R) at position 336 is not substituted with Lys (K). In a particular embodiment, the Arg at position 336 is substituted with Asp (D), Glu (E), Asn (N), or Gln (Q). In a particular embodiment, the Arg at position 336 is substituted with Asn (N) or Gln (Q). In a particular embodiment, the Arg at position 336 is substituted with Gln (Q).

In certain embodiments, the Factor VIII variants comprise a mutation at position 562. In a particular embodiment, the Arg (R) at position 562 is not substituted with Lys (K). In a particular embodiment, the Arg at position 562 is substituted with Asp (D), Glu (E), Asn (N), or Gln (Q). In a particular embodiment, the Arg at position 562 is substituted with Asn (N) or Gln (Q). In a particular embodiment, the Arg at position 562 is substituted with Gln (Q).

In certain embodiments, the Factor VIII variants comprise a mutation at position 372. In a particular embodiment, the Arg (R) at position 372 is not substituted with Lys (K). In a particular embodiment, the Arg at position 372 is substituted with Asp (D), Glu (E), Asn (N), or Gln (Q). In a particular embodiment, the Arg at position 372 is substituted with Asn (N) or Gln (Q). In a particular embodiment, the Arg at position 372 is substituted with Gln (Q).

In certain embodiments, the Factor VIII variants comprise a mutation at position 740. In a particular embodiment, the Arg (R) at position 740 is not substituted with Lys (K). In a particular embodiment, the Arg at position 740 is substituted with Asp (D), Glu (E), Asn (N), or Gln (Q). In a particular embodiment, the Arg at position 740 is substituted with Asn (N) or Gln (Q). In a particular embodiment, the Arg at position 740 is substituted with Gln (Q).

In certain embodiments, the Factor VIII variants comprise a mutation at position 1689. In a particular embodiment, the Arg (R) at position 1689 is not substituted with Lys (K). In a particular embodiment, the Arg at position 1689 is substituted with Asp (D), Glu (E), Asn (N), or Gln (Q). In a particular embodiment, the Arg at position 1689 is substituted with Asn (N) or Gln (Q). In a particular embodiment, the Arg at position 1689 is substituted with Gln (Q).

As stated hereinabove, the FVIII variant of the instant invention may be human. In a particular embodiment, the FVIII variant of the instant invention has at least 75%, 80%, 85%, 90%, 95%, 97%, 99%, or 100% homology (identity) with SEQ ID NO: 1 (or fragment or domain thereof or an activated FVIII fragment thereof), particularly at least 90%, 95%, 97%, 99%, or 100% homology (identity). In a particular embodiment, the FVIII variant comprises an amino acid sequence having at least 75%, 80%, 85%, 90%, 95%, 97%, 99%, or 100% homology (identity), particularly at least 90%, 95%, 97%, 99%, or 100% homology (identity), with amino acids 1-740 of SEQ ID NO: 1 (or fragment or domain thereof or an activated FVIII fragment thereof) and an amino acid sequence having at least 75%, 80%, 85%, 90%, 95%, 97%, 99%, or 100% homology (identity), particularly at least 90%, 95%, 97%, 99%, or 100% homology (identity) with amino acids 1649-2332 or 1690-2332 of SEQ ID NO: 1 (or fragment or domain thereof or an activated FVIII fragment thereof). The homology (identity) percentages above exclude the substitutions at position 336, 562, 372, 740, and/or 1689.

The FVIII variants of the instant invention may also be post-translationally modified. The FVIII variants may be post-translationally modified in a cell (particularly a human cell) or in vitro.

In a particular embodiment, the FVIII variants of the invention have increased resistance to cleavage and/or inactivation by a serine protease compared to wild-type FVIII.

Nucleic acid molecules encoding the above FVIII variants (or fragments or domains thereof or activated fragments thereof) are also encompassed by the instant invention. Nucleic acid molecules encoding the variants may be prepared by any method known in the art. The nucleic acid molecules may be maintained in any convenient vector, particularly an expression vector.

Compositions comprising at least one FVIII variant and at least one carrier (e.g., pharmaceutically acceptable carrier) are also encompassed by the instant invention. In a particular embodiment, the FVIII is isolated and/or substantially pure within the composition. Compositions comprising at least one FVIII variant nucleic acid molecule and at least one carrier are also encompassed by the instant invention. Except insofar as any conventional carrier is incompatible with the variant to be administered, its use in the pharmaceutical composition is contemplated. In a particular embodiment, the carrier is a pharmaceutically acceptable carrier for intravenous administration.

Nucleic acid molecules encoding the variants of the invention may be prepared by using recombinant DNA technology methods. The availability of nucleotide sequence information enables preparation of isolated nucleic acid molecules of the invention by a variety of means. For example, nucleic acid sequences encoding a variant may be isolated from appropriate biological sources using standard protocols well known in the art.

Nucleic acids of the present invention may be maintained as RNA or DNA in any convenient cloning vector. In a particular embodiment, clones are maintained in a plasmid cloning/expression vector, which is propagated in a suitable host cell (e.g., E. coli). Alternatively, the nucleic acids may be maintained in a vector suitable for expression in mammalian cells. In cases where post-translational modification affects variant function, it is preferable to express the molecule in mammalian cells, particularly human cells.

FVIII variant encoding nucleic acid molecules of the invention include DNA, cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention. Such oligonucleotides are useful as probes for detecting variant expression.

The FVIII variants of the present invention may be prepared in a variety of ways, according to known methods. The protein may be purified from appropriate sources (e.g., transformed bacterial or animal (e.g., mammalian or human) cultured cells or tissues which express FVIII variants), for example, by immunoaffinity purification or cation exchange chromatography purification. The availability of nucleic acid molecules encoding the variants enables production of the variants using in vitro expression methods known in the art. For example, a cDNA or gene may be cloned into an appropriate in vitro transcription vector followed by cell-free translation in a suitable cell-free translation system, such as wheat germ or rabbit reticulocyte lysates. In vitro transcription and translation systems are commercially available.

Alternatively, larger quantities of variant may be produced by expression in a suitable prokaryotic or eukaryotic expression system. For example, part or all of a DNA molecule encoding the FVIII variant may be inserted into a plasmid vector adapted for expression in a bacterial cell, such as E. coli, or a mammalian cell (particularly a human cell) such as CHO, BHK, or HeLa cells. Alternatively, tagged fusion proteins comprising the variant can be generated. Such variant-tagged fusion proteins are encoded by part or all of a DNA molecule, ligated in the correct codon reading frame to a nucleotide sequence encoding a portion or all of a desired polypeptide tag which is inserted into a plasmid vector adapted for expression in a bacterial cell, such as E. coli or a eukaryotic cell, such as, but not limited to, yeast and mammalian cells, particularly human cells. Vectors such as those described above comprise the regulatory elements necessary for expression of the DNA in the host cell positioned in such a manner as to permit expression of the DNA in the host cell. Such regulatory elements required for expression include, but are not limited to, promoter sequences, transcription initiation sequences, and enhancer sequences.

FVIII variant proteins, produced by gene expression in a recombinant prokaryotic or eukaryotic system (particularly human) may be purified according to methods known in the art. In a particular embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise, without limitation, the FLAG epitope, GST or the hemagglutinin epitope. Such methods are commonly used by skilled practitioners.

FVIII variant proteins, prepared by the aforementioned methods, may be analyzed according to standard procedures. For example, such proteins may be subjected to amino acid sequence analysis, according to known methods.

As discussed above, a convenient way of producing a polypeptide according to the present invention is to express nucleic acid encoding it, by use of the nucleic acid in an expression system. A variety of expression systems of utility for the methods of the present invention are well known to those of skill in the art.

Accordingly, the present invention also encompasses a method of making a polypeptide. In certain embodiments, the method includes expression from nucleic acid encoding the polypeptide. This may conveniently be achieved by culturing a host cell, containing such a vector, under appropriate conditions which cause or allow production of the polypeptide. Polypeptides may also be produced in in vitro systems, such as in reticulocyte lysates.

FVIII variant proteins and nucleic acids of the instant invention may be used, for example, as therapeutic and/or prophylactic agents which modulate the blood coagulation cascade. The FVIII variant proteins and nucleic acids of the instant invention may be administered in a therapeutically effective amount to modulate (e.g., increase) hemostasis and/or form a clot and/or stop or inhibit bleeding or aberrant bleeding. It is demonstrated herein that the FVIII variants possess superior properties and can provide effective hemostasis.

In a particular embodiment of the present invention, FVIII variants may be administered to a patient via infusion in a biologically compatible carrier, e.g., via injection or intravenous injection. The FVIII variants of the invention may optionally be encapsulated into liposomes or mixed with other phospholipids or micelles to increase stability of the molecule. FVIII variants may be administered alone or in combination with other agents known to modulate hemostasis (e.g., vFW, Factor IX, Factor IXa, etc.). An appropriate composition in which to deliver the FVIII variant may be determined by a medical practitioner upon consideration of a variety of physiological variables, including, but not limited to, the patient's condition and hemodynamic state. A variety of compositions well suited for different applications and routes of administration are well known in the art and are described hereinbelow.

The preparation containing the FVIII variants may contain a physiologically acceptable matrix and is formulated as a pharmaceutical preparation. The preparation can be formulated using substantially known methods, it can be mixed with a buffer containing salts, such as NaCl, CaCl₂, and amino acids, such as glycine and/or lysine, and in a pH range from 6 to 8. Until needed, the purified preparation containing the FVIII variant can be stored in the form of a finished solution or in lyophilized or deep-frozen form. In a particular embodiment, the preparation is stored in lyophilized form and is dissolved into a visually clear solution using an appropriate reconstitution solution. Alternatively, the preparation according to the present invention can also be made available as a liquid preparation or as a liquid that is deep-frozen. The preparation according to the present invention may be especially stable, i.e., it can be allowed to stand in dissolved form for a prolonged time prior to application.

The preparation according to the present invention can be made available as a pharmaceutical preparation with the FVIII variant in the form of a one-component preparation or in combination with other factors in the form of a multi-component preparation.

Prior to processing the purified protein into a pharmaceutical preparation, the purified protein may be subjected to the conventional quality controls and fashioned into a therapeutic form of presentation. In particular, during the recombinant manufacture, the purified preparation may be tested for the absence of cellular nucleic acids as well as nucleic acids that are derived from the expression vector.

Another feature of this invention relates to making available a preparation which contains a FVIII variant with a high stability and structural integrity and which, in particular, is free from inactive FVIII intermediates and/or proteolytic degradation products and by formulating it into an appropriate preparation.

The pharmaceutical preparation may contain, as an example, dosages of between about 1-1000 μg/kg, about 10-500 μg/kg, about 10-250 μg/kg, or about 10-100 μg/kg. In a particular embodiment, the pharmaceutical protein preparation may comprise a dosage of between 30-100 IU/kg (e.g., as a single daily injection or up to 3 times or more/day). Patients may be treated immediately upon presentation at the clinic with a bleed or prior to the delivery of cut/wound causing a bleed. Alternatively, patients may receive a bolus infusion every one to three, eight, or twelve hours or, if sufficient improvement is observed, a once daily infusion of the FVIII variant described herein.

FVIII variant-encoding nucleic acids may be used for a variety of purposes in accordance with the present invention such as gene therapy and/or gene editing. In a particular embodiment of the invention, a nucleic acid delivery vehicle (e.g., an expression vector such as a viral vector) for modulating blood coagulation is provided wherein the expression vector comprises a nucleic acid sequence coding for a FVIII variant as described herein. Administration of the FVIII variant-encoding expression vectors to a patient results in the expression of the FVIII variant which serves to alter the coagulation cascade. In accordance with the present invention, a FVIII variant encoding nucleic acid sequence may encode a variant polypeptide as described herein whose expression increases hemostasis. In a particular embodiment, the nucleic acid sequence encodes a human FVIII variant.

Expression vectors comprising FVIII variant nucleic acid sequences may be administered alone, or in combination with other molecules useful for modulating hemostasis. According to the present invention, the expression vectors or combination of therapeutic agents may be administered to the patient alone or in a pharmaceutically acceptable or biologically compatible composition.

In a particular embodiment of the invention, the expression vector comprising nucleic acid sequences encoding the FVIII variant is a viral vector or non-viral vector. Viral vectors which may be used in the present invention include, but are not limited to, adenoviral vectors (with or without tissue specific promoters/enhancers), adeno-associated virus (AAV) vectors of any serotype (e.g., AAV-1 to AAV-12, particularly AAV-2, AAV-5, AAV-7, and AAV-8) and hybrid AAV vectors, lentivirus vectors and pseudo-typed lentivirus vectors (e.g., Ebola virus, vesicular stomatitis virus (VSV), and feline immunodeficiency virus (FIV)), herpes simplex virus vectors, vaccinia virus vectors, and retroviral vectors. Examples of non-viral means include, without limitation, gene delivery by lipid nanoparticles. In a particular embodiment, the vector is an adeno-associated virus (AAV) vector. In a particular embodiment, the vector is a lentiviral vector.

The instant invention also encompasses gene therapy methods (e.g., for modulating hemostasis) with the FVIII variants of the instant invention. In a particular embodiment of the present invention, methods are provided for the administration of a viral vector comprising nucleic acid sequences encoding a FVIII variant. Viral (e.g., AAV) vectors of utility in the methods of the present invention preferably include at least the essential parts of viral (e.g., AAV) vector DNA. As described herein, expression of a FVIII variant following administration of such a viral (e.g., AAV) vector serves to modulate hemostasis, particularly to enhance the procoagulation activity of the protease.

Recombinant viral (e.g., AAV) vectors have found broad utility for a variety of gene therapy applications. Their utility for such applications is due largely to the high efficiency of in vivo gene transfer achieved in a variety of organ contexts.

AAV particles may be used to advantage as vehicles for adequate gene delivery. Such virions possess a number of desirable features for such applications, including: structural features related to being a double stranded DNA nonenveloped virus and biological features such as a tropism for the human respiratory system and gastrointestinal tract. Moreover, AAV are known to infect a wide variety of cell types in vivo and in vitro by receptor-mediated endocytosis. Attesting to the overall safety of AAV vectors, infection with AAV leads to a minimal disease state in humans comprising mild flu-like symptoms.

Viral (e.g., AAV) genomes are well suited for use as gene therapy vehicles because they can accommodate the insertion of foreign DNA following the removal of viral genes essential for replication and/or nonessential regions. Such substitutions render the viral vector impaired with regard to replicative functions and infectivity. Many viruses (e.g., AAV) have been used as vectors for gene therapy and for expression of heterologous genes.

It is desirable to introduce a vector that can provide, for example, multiple copies of a desired gene and hence greater amounts of the product of that gene. Improved viral (e.g., AAV) vectors and methods for producing these vectors have been described (e.g., Penn Vector Core; addgene; etc.).

For some applications, an expression construct may further comprise regulatory elements which serve to drive expression in a particular cell or tissue type and/or constitutively. Such regulatory elements are known to those of skill in the art. The incorporation of tissue specific regulatory elements in the expression constructs of the present invention provides for at least partial tissue tropism for the expression of the variant or functional fragments thereof. In certain embodiments, a constitutive promoter (e.g., cytomegalovirus (CMV) promoter) may be used. Hematopoietic or liver specific promoters may also be used.

AAV for recombinant gene expression have been produced in human cells (e.g., the human embryonic kidney cell line 293). Briefly, AAV vectors are typically engineered from wild-type AAV, a single-stranded DNA virus that is non-pathogenic. The parent virus is non-pathogenic, the vectors have a broad host range, and they can infect both dividing and non-dividing cells. The vector is typically engineered from the virus by deleting the rep and cap genes and replacing these with the transgene of interest under the control of a specific promoter. For recombinant AAV preparation, the upper size limit of the sequence that can be inserted between the two ITRs is about 4.7 kb. Plasmids expressing a FVIII variant under the control of a promoter (e.g., the CMV promoter/enhancer) and a second plasmid supplying adenovirus helper functions along with a third plasmid containing the rep and cap genes (e.g., AAV-2 rep and cap genes) may be used to produce AAV vectors (e.g., AAV-2 vectors). Other AAV serotype cap genes (e.g., AAV-1, AAV-6, or AAV-8 cap genes) may be expressed with other serotype rep genes and ITRs (e.g., AAV-2 rep gene and ITRs) to produce different vectors (e.g., Gao et al. (2002) Proc. Natl. Acad. Sci. USA 99:11854-11859; Xiao et al., (1999) J. Virol. 73:3994-4003; Arruda et al., (2004) Blood 103:85-92). AAV vectors may be purified by repeated CsCl density gradient centrifugation and the titer of purified vectors determined by quantitative dot-blot hybridization. In a particular embodiment, vectors may be prepared by the Vector Core at The Children's Hospital of Philadelphia.

Also included in the present invention is a method for modulating hemostasis comprising providing cells of an individual with a nucleic acid delivery vehicle encoding a FVIII variant and allowing the cells to grow under conditions wherein the FVIII variant is expressed.

From the foregoing discussion, it can be seen that FVIII variants and FVIII variant encoding nucleic acid molecules (e.g., FVIII variant expressing nucleic acid vectors) may be used in the treatment of disorders associated with aberrant blood coagulation. The instant invention encompasses methods of increasing FVIII expression (e.g., in vivo), increasing plasma concentration of FVIII, and/or increasing steady-state levels of FVIII (e.g., by introducing at least one mutation at position 336, 562, 372, 740, and/or 1689 as described herein). The FVIII variants of the instant invention have demonstrated increased expression, increased plasma concentrations, and increased steady-state levels compared to wild-type FVIII.

FVIII variant encoding nucleic acid molecules (e.g., expression vectors) of the present invention may be incorporated into pharmaceutical compositions that may be delivered to a subject, so as to allow production of a biologically active protein (e.g., a FVIII variant) or by inducing expression of the FVIII variant in vivo by gene- and or cell-based therapies or by ex vivo modification/transduction of the patient's or donor's cells. The FVIII variant encoding nucleic acid molecules may be used for gene addition or gene editing to express the FVIII variants of the instant invention. In a particular embodiment of the present invention, pharmaceutical compositions comprising sufficient genetic material to enable a recipient to produce a therapeutically effective amount of a FVIII variant can influence hemostasis in the subject. Alternatively, as discussed above, an effective amount of the FVIII variant may be directly infused into a patient in need thereof. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents (e.g., co-factors) which influence hemostasis.

In particular embodiments, the compositions (e.g., pharmaceutical compositions) of the instant invention also contain a pharmaceutically acceptable carrier. Such carriers include any pharmaceutical agent that does not itself induce an immune response harmful to the individual receiving the composition, and which may be administered without undue toxicity. Pharmaceutically acceptable carriers include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of FVIII variants or FVIII variant encoding nucleic acids (e.g., vectors), such labeling could include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques and guidance provided in the present invention. Therapeutic doses will depend on, among other factors, the age and general condition of the subject, the severity of the aberrant blood coagulation phenotype, and the strength of the control sequences regulating the expression levels of the variant polypeptide. Thus, a therapeutically effective amount in humans will fall in a relatively broad range that may be determined by a medical practitioner based on the response of an individual patient to vector-based variant treatment.

The FVIII variants, alone or in combination with other agents, may be directly infused into a patient in an appropriate biological/pharmaceutical carrier as described hereinabove. Expression vectors of the present invention comprising nucleic acid sequences encoding variant or functional fragments thereof, may be administered to a patient by a variety of means (see below) to achieve and maintain a prophylactically and/or therapeutically effective level of the variant polypeptide. One of skill in the art could readily determine specific protocols for using the variant encoding expression vectors of the present invention for the therapeutic treatment of a particular patient.

FVIII variants and/or FVIII variant encoding nucleic acids (e.g., AAV vectors) of the present invention may be administered to a patient by any means known. Direct delivery of the pharmaceutical compositions in vivo may generally be accomplished via injection using a conventional syringe, although other delivery methods such as convection-enhanced delivery are envisioned. In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include oral and pulmonary administration, suppositories, and transdermal applications. In certain embodiments, the FVIII is administered by injection (e.g., to the bloodstream). In certain embodiments, the FVIII variant encoding nucleic acids (e.g., AAV vectors) is administered by injection (e.g., to the bloodstream or liver). A clinician specializing in the treatment of patients with blood coagulation disorders may determine the optimal route for administration of the vectors (e.g., AAV vectors) comprising variant nucleic acid sequences based on a number of criteria, including, but not limited to: the condition of the patient and the purpose of the treatment (e.g., reduced blood coagulation).

The present invention also encompasses vectors (e.g., viral vectors or AAV vectors) comprising a nucleic acid sequence encoding a FVIII variant. Also provided are lentiviruses or pseudo-typed lentivirus vectors comprising a nucleic acid sequence encoding a FVIII variant. Also encompassed are naked plasmid or expression vectors comprising a nucleic acid sequence encoding a FVIII variant.

### Definitions

The following definitions are provided to facilitate an understanding of the present invention.

The singular forms “a,” “an,” and “the” include plural referents unless the context clearly dictates otherwise.

The phrase “hemostasis related disorder” refers to bleeding disorders such as, without limitation, hemophilia A, hemophilia B, hemophilia A and B patients, hemophilia with inhibitory antibodies, deficiencies in at least one coagulation factor (e.g., Factors VII, VIII, IX, X, XI, V, XII, II, and/or von Willebrand factor, particularly Factor VIII), combined FV/FVIII deficiency, vitamin K epoxide reductase C1 deficiency, gamma-carboxylase deficiency, bleeding associated with trauma or injury, thrombosis, thrombocytopenia, stroke, coagulopathy (hypocoagulability), disseminated intravascular coagulation (DIC), over-anticoagulation associated with heparin, low molecular weight heparin, pentasaccharide, warfarin, or small molecule antithrombotics (e.g., FXa inhibitors); and platelet disorders such as, Bernard Soulier syndrome, Glanzman thromblastemia, and storage pool deficiency. In a particular embodiment, the term “hemostasis related disorder” refers to bleeding disorders characterized by excessive and/or uncontrolled bleeding (e.g., a disorder which can be treated with a procoagulant). In a particular embodiment, the hemostasis related disorder is hemophilia. In a particular embodiment, the hemostasis related disorder is hemophilia A.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote. With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

With respect to protein, the term “isolated protein” is sometimes used herein. This term may refer to a protein produced by expression of an isolated nucleic acid molecule of the invention. Alternatively, this term may refer to a protein which has been sufficiently separated from other proteins with which it would naturally be associated (e.g., so as to exist in “substantially pure” form). “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “vector” refers to a carrier nucleic acid molecule (e.g., RNA or DNA) into which a nucleic acid sequence can be inserted for introduction into a host cell where it will be replicated. An “expression vector” is a specialized vector that contains a gene or nucleic acid sequence with the necessary regulatory regions (e.g., promoter) needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.), particularly at least 75% by weight, or at least 90-99% or more by weight of the compound of interest. Purity may be measured by methods appropriate for the compound of interest (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “subject” refers to an animal, particularly a mammal, particularly a human.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., aberrant bleeding) resulting in a decrease in the probability that the subject will develop the condition.

A “therapeutically effective amount” of a compound or a pharmaceutical composition refers to an amount effective to prevent, inhibit, treat, and/or lessen the symptoms of a particular disorder or disease.

The following example is provided to illustrate various embodiments of the present invention. The example is illustrative and is not intended to limit the invention in any way.

### EXAMPLE

Coagulation maintains an enclosed circulatory system and functions as an enzymatic cascade in which the catalytic activity of the associated enzymes is conferred by chymotrypsin-like serine proteases wherein the catalytic triad cleaves characteristic Arg residues of the associated substrate. All major enzymes of the coagulation fall into this category and include thrombin (IIa), FXIa, FIXa, FXa, FVIIa, plasmin, activated protein C (APC), tissue plasminogen activator, etc. Coagulation factor VIII (FVIII) is the deficient protein in hemophilia A (HA) and circulates in plasma. FVIII functions as a cofactor within the intrinsic tenase enzyme complex to generate activated factor X (FXa) and propagate clot formation. FVIII is activated by serine protease cleavage by thrombin (Ha) as well as, to a lesser extent, cleavage by another serine proteases, including FIXa and FXa. There are three described IIa cleavage sites (FIG. 1B). Once activated, FVIIIa is inactivated by either spontaneous dissociation of the A2 domain that is maintained in the FVIIIa heterotrimer by weak electrostatic interaction or serine protease cleavage by APC that, together with its cofactor protein S (PS), inactivate FVIIIa by cleaving R336 and R562 (FIG. 1B).

Despite progress in adeno-associated virus (AAV) mediated factor VIII (FVIII) gene transfer for hemophilia A (HA), durable and sustained FVIII expression sufficient to ameliorate bleeding in all participants is an unrealized goal (Samelson-Jones, et al., Annu. Rev. Med. (2023) 74:231-247). Following the success of using the gain of function FIX-Padua variant in hemophilia B gene therapy, it was hypothesized that use of an enhanced hemostatic function FVIII would similarly improve HA gene therapy by: 1) permitting use of lower vector doses, thereby overcoming dose-dependent safety and efficacy limitations and 2) permit efficacy at lower FVIII antigen. A FVIII variant resistant to activated protein C (APC) cleavage (FVIII-R336Q/R562Q or FVIII-QQ) has been determined to have normal specific activity and 4-5 fold improved hemostatic function in recombinant protein experiments (Wilhelm et al., Blood (2021) 137(18):2532-2543; WO 2021/113800).

Herein, the possible clinical translation of FVIII-QQ for HA AAV-mediated gene therapy was evaluated by measuring the hemostatic benefit and potential prothrombotic and immunological risk.

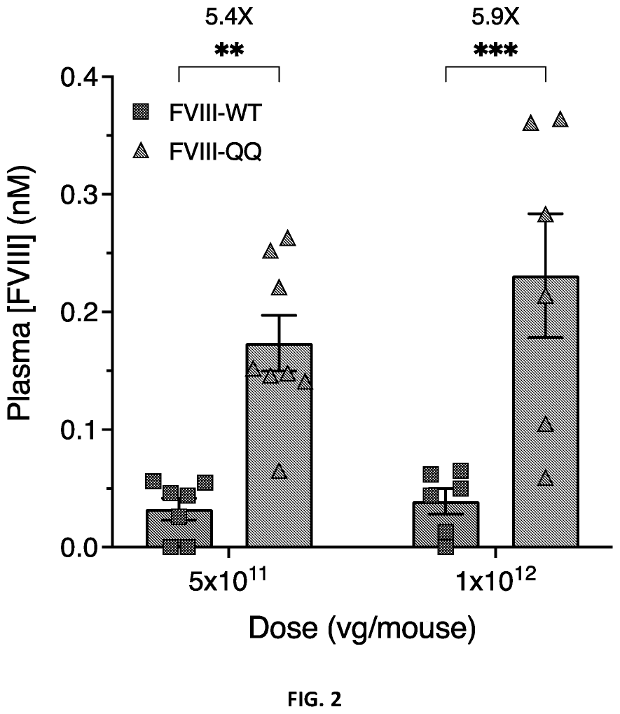

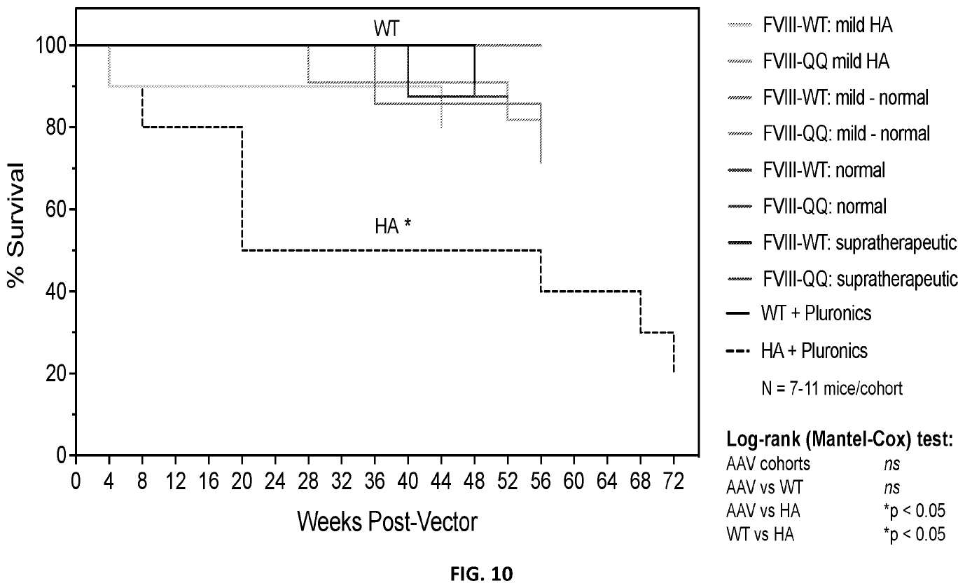

First, hemophilia A (HA) mice were treated with AAV vectors which expressed FVIII-QQ or wild-type FVIII (FVIII-WT). The AAV vectors were identical except for modification of the referenced R336 and R562 residues. Mice were treated with either 5×10¹¹ or 1×10¹² vector genomes (vg)/mouse. As seen in FIG. 2, FVIII-QQ plasma concentrations were significantly higher (~5-fold higher) than FVIII-WT despite equal AAV vector dosing. Further, survival studies in HA/CD4KO and WT/CD4KO mice treated with AAV vectors to express human FVIII-WT and FVIII-QQ in the range of mild HA, normal, and supratherapeutic FVIII levels demonstrate no significant differences in survival at 7-11 month or D-dimer—a fibrin degradation product present in the blood after a blood clot is degraded by fibrinolysis (FIG. 10). HA immune competent mice treated with either 6 weekly doses of hFVIII-WT or hFVIII-QQ protein (0.2 μg) or AAV vector to express hFVIII-QQ or hFVIII-WT had no significant differences in inhibitor titers.

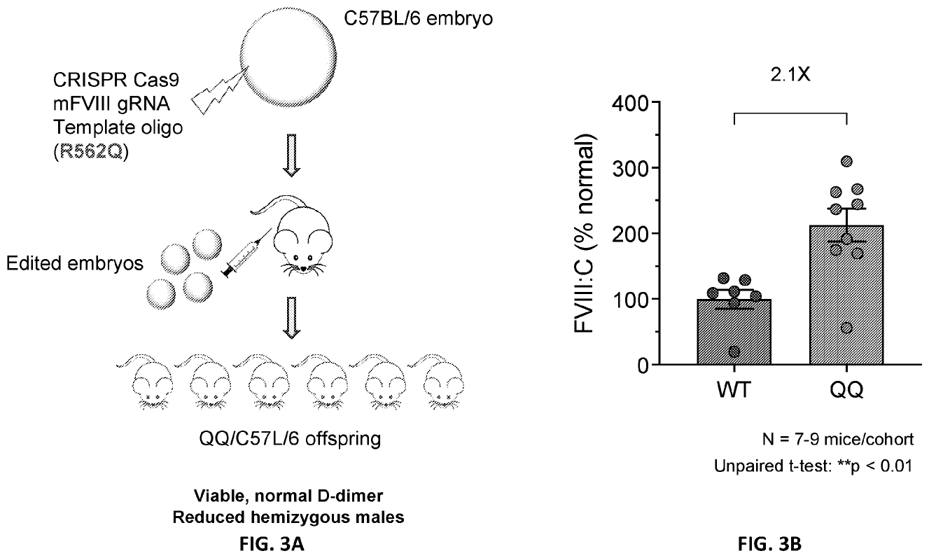

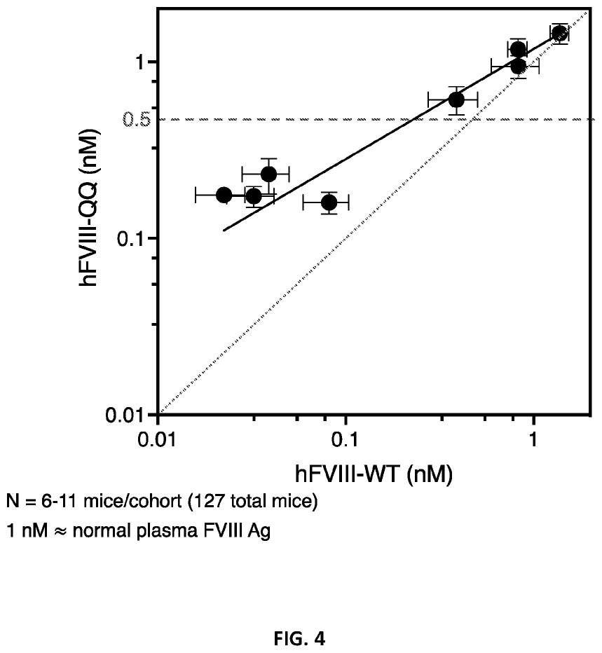

Additionally, FVIII-QQ mice were generated using CRISPR/Cas9 to introduce R562Q (wild-type mice are already Q336) for endogenous mouse FVIII-QQ expression (FIG. 3A). Mice were viable, fertile, normal weight, and D-dimers did not significantly differ from WT mice. In contrast, FVIII antigen was ~2-fold higher in FVIII-QQ mice than WT mice (FIG. 3B). Thus, increased FVIII-QQ was demonstrated in 2 model systems: 1) gene therapy, and 2) endogenous expression following genome editing. This was particularly pronounced at plasma concentrations <0.5 nM (normal FVIII concentration is approximately 1 nM) (FIG. 4). Due to enhanced FVIII-QQ hemostatic function, expression at <0.5 nM could be targeted for clinical translation.

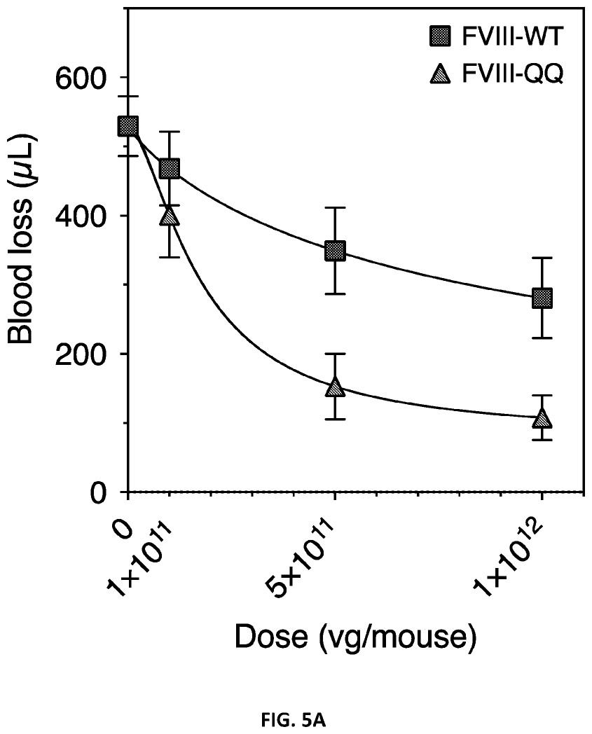

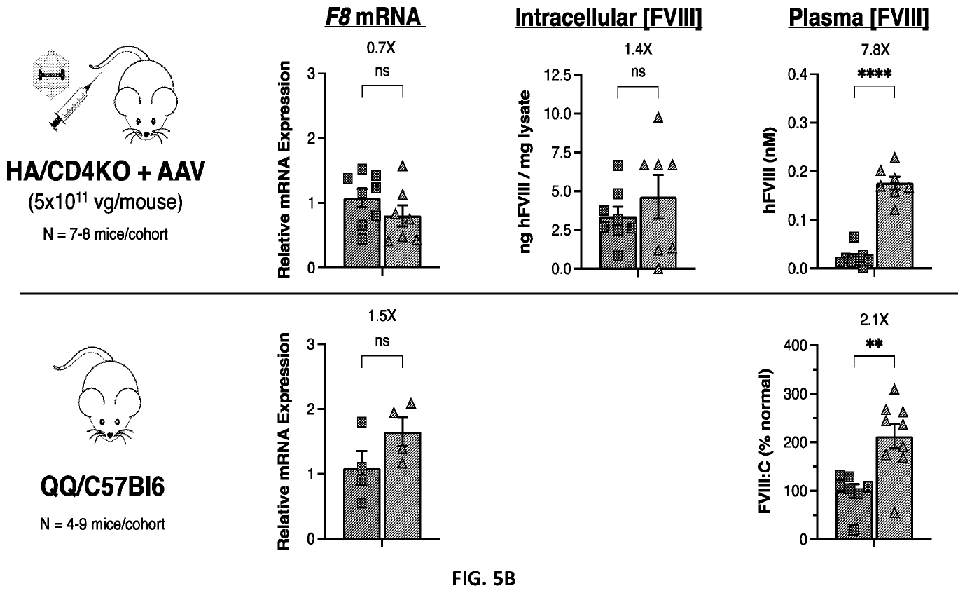

To evaluate hemostasis, HA/CD4KO mice were treated with the same AAV vector construct differing by 2 amino acids at 3 different vector doses (n=6-12 mice/cohort) to express hFVIII-QQ or hFVIII-WT and underwent tail clip challenge (FIG. 5A). Estimated EC₅₀ and EC₈₀ vector doses required to normalize blood loss to wild-type mice, were 5 and 10-fold lower, respectively, for FVIII-QQ versus FVIII-WT supporting the hemostatic benefit of using the FVIII-QQ transgene (Table 1). Interestingly, across 3 different vector constructs differing only by FVIII codon optimization, FVIII-QQ antigen values were observed to be 2.5 to 5-fold higher than FVIII-WT.

TABLE 1
Hemostatic effect.

| | Tail Clip EC₅₀ | Tail Clip EC₈₀ |
|---|---|---|
| | (vg/mouse) | (vg/mouse) |
| FVIII-WT | 8.18 × 10¹¹ | 4.17 × 10¹² |
| FVIII-QQ | 1.73 × 10¹¹ | 4.19 × 10¹¹ |

Analysis of liver tissue demonstrated no differences in FVIII-WT versus FVIII-QQ mRNA, indicating the mechanism of higher FVIII-QQ steady-state expression occurs post translationally (FIG. 5B). There was also no statistical difference in intracellular FVIII, indicating the mechanism occurs in plasma (FIG. 5B). Indeed, the difference in FVIII-QQ and FVIII-WT post-AAV was only observed in plasma (FIG. 5B). The same trend was observed in the CRISPR/cas9 generated FVIII-QQ mouse model versus wild type littermate controls (FIG. 5B).

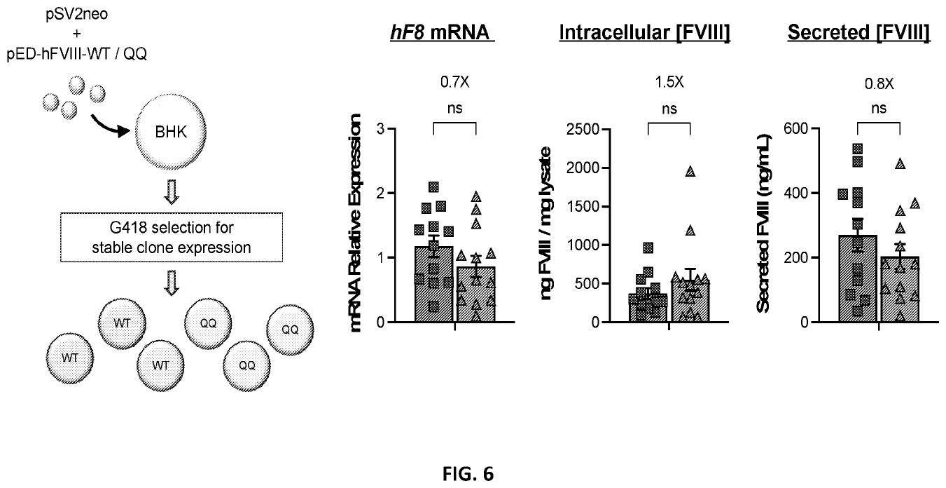

Additionally, stable BHK cell lines expressing either FVIII-WT or FVIII-QQ (n=12 lines/cohort) were generated. FVIII mRNA, intracellular FVIII, and secreted FVIII were measured in conditioned media. As seen in FIG. 6, none of these values significantly differed between FVIII-WT or FVIII-QQ. Collectively, this data supports the difference in plasma FVIII concentrations observed between FVIII-WT and FVIII-QQ is due to a process occurring post transcriptionally, post translationally, and in plasma.

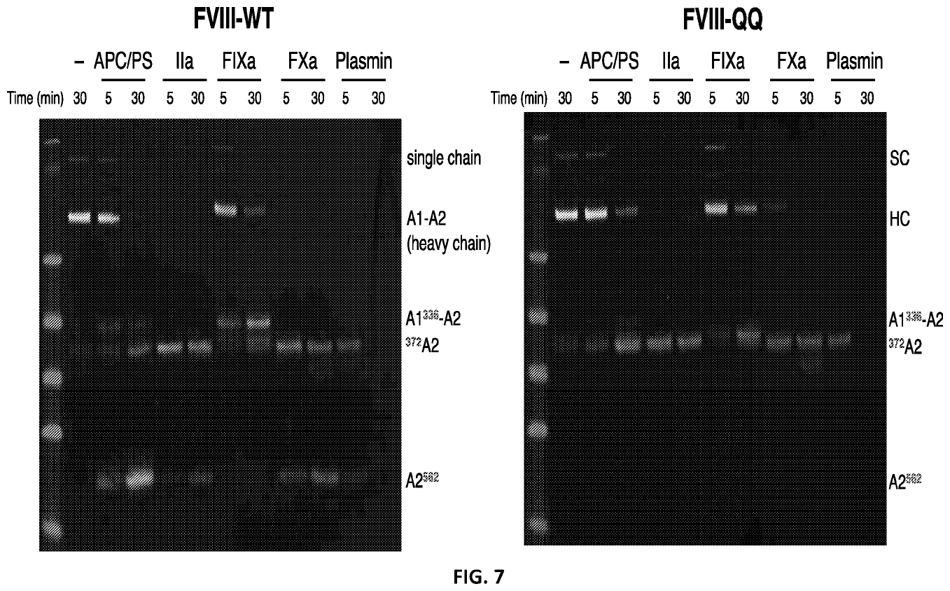

Next, recombinant FVIII-QQ and FVIII-WT were incubated with a panel of serine proteases (APC, FIXa, FXa, thrombin (IIa), and plasmin) for 5-30 minutes. By Western Blot analysis, all serine proteases cleaved at R336 or R562 in FVIII-WT (FIG. 7). In contrast, no such cleavage fragments were seen with FVIII-QQ (FIG. 7). Thus, it is clear that protease attack at these cleavage sites occurs more than previously appreciated and by more serine proteases.

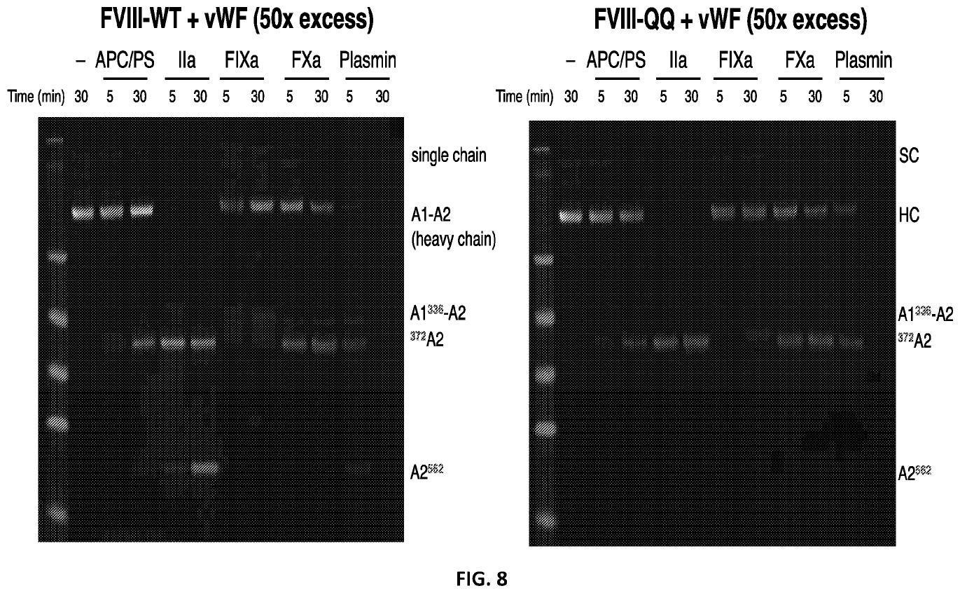

Similar results, albeit with lesser cleavage, was observed in the presence of 50-fold molar excess of Von Willebrand factor (vWF) (FIG. 8). FVIII circulates in plasma bound to vWF in a sub-nanomal affinity interaction (kd~100-900 pM) and vWF is present in 50-fold molar excess of FVIII in plasma. Notably, in vitro binding assays demonstrate that rFVIII-QQ has the same binding affinity for vWF as wild-type. Collectively, these data indicate that avoidance of serine protease cleavage permits higher steady-state FVIII-QQ expression. FVIII-QQ was more proteolytically stable when bound to vWF.

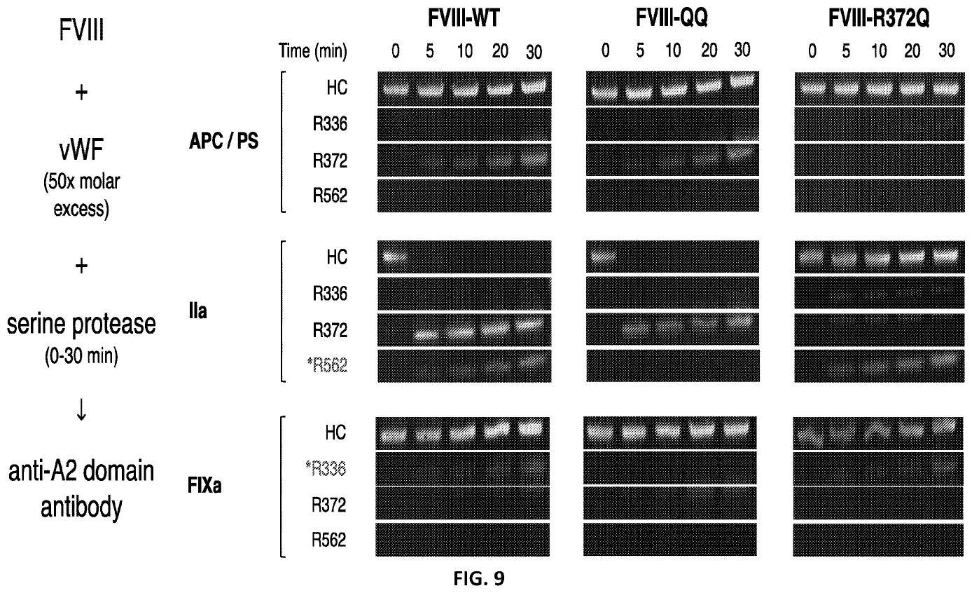

These observations are unlikely to be specific to the R336 and R562 residues. Specifically, it was demonstrated that thrombin (IIa) as well as multiple serine proteases can also cleave R372 of FVIII (FIGS. 7 and 8), which is a significant cleavage residue to permit activation to FVIIIa. The ability of a FVIII variant with a R372Q mutation was tested. As seen in FIG. 9, the FVIII-R372Q variant could not be cleaved at 372. Further, FVIII-WT and FVIII-R372Q can be cleaved at R336 and/or R562 in the presence of vWF.

The use of FVIII-QQ transgene expression does not demonstrate safety concerns and confers hemostatic benefit over FVIII-WT via unexpectedly superior: 1) improved hemostatic function and 2) higher steady-state expression. Enhanced steady state FVIII-QQ expression relative to FVIII-WT was observed across multiple model systems.

Collectively, these data support introducing mutations at arginine serine protease cleavage sites of FVIII to make the FVIII protein resistant to proteolytic cleavage, thereby permitting higher steady-state plasma FVIII concentrations in the context of gene therapy and/or gene editing, which imparts a therapeutic advantage. This is not specific to APC cleavage or the R336 and R562 cleavage sites as it is demonstrated herein that multiple serine proteases can cleave at the described FVIII APC cleavage sites (R336 and R562).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An adeno-associated virus vector comprising a nucleic acid encoding a Factor VIII (FVIII) variant wherein at least one cleavage site for a serine protease has been mutated.

2. The adeno-associated virus vector of claim 1, wherein the serine protease is selected from the group consisting of APC, FIXa, FXa, thrombin (IIa), and plasmin

3. The adeno-associated virus vector of claim 1, wherein said FVIII variant comprises at least one mutation at position 336, 562, 372, 740, and/or 1689.

4. The adeno-associated virus vector of claim 1, wherein said FVIII variant comprises a mutation at positions 336 and 562.

5. The adeno-associated virus vector of claim 1, wherein said FVIII variant comprises a mutation at positions 336 and 562 and at least one mutation at position 372, 740, and/or 1689.

6. The adeno-associated virus vector of claim 3, wherein the Arg at position 336, 562, 372, 740, and/or 1689 is substituted with Gln.

7. The adeno-associated virus vector of any one of claims 1-6, wherein said FVIII variant lacks the B domain or the B domain has been replaced by a peptide linker.

8. The adeno-associated virus vector of any one of claims 1-7, wherein said FVIII variant comprises amino acids 1-740 and 1649-2332 of SEQ ID NO: 1.

9. The adeno-associated virus vector of any one of claims 1-8, wherein said FVIII variant comprises amino acids 1-740 and 1690-2332 of SEQ ID NO: 1.

10. A composition comprising at least one adeno-associated virus vector of any one of claims 1-9 and at least one pharmaceutically acceptable carrier.

11. A method for treatment of a hemostasis related disorder in a patient in need thereof comprising administering a therapeutically effective amount of the adeno-associated virus vector of any one of claims 1-9 in a pharmaceutically acceptable carrier to said patient.

12. The method of claim 11, wherein said hemostasis related disorder is hemophilia.

13. A method for reducing blood loss in a patient in need thereof comprising administering a therapeutically effective amount of the adeno-associated virus vector of any one of claims 1-9 in a pharmaceutically acceptable carrier to said patient.

14. A Factor VIII (FVIII) variant wherein at least one cleavage site for a serine protease has been mutated.

15. The FVIII variant of claim 14, wherein the serine protease is selected from the group consisting of APC, FIXa, FXa, thrombin (IIa), and plasmin

16. The FVIII variant of claim 14, wherein said FVIII variant comprises at least one mutation at position 336, 562, 372, 740, and/or 1689.

17. The FVIII variant of claim 14, wherein said FVIII variant comprises a mutation at positions 336 and 562.

18. The FVIII variant of claim 14, wherein said FVIII variant comprises a mutation at positions 336 and 562 and at least one mutation at position 372, 740, and/or 1689.

19. The FVIII variant of claim 14, wherein the Arg at position 336, 562, 372, 740, and/or 1689 is substituted with Gln.

20. The FVIII variant of any one of claims 14-19, wherein the FVIII variant lacks the B domain or the B domain has been replaced by a peptide linker.

21. The FVIII variant of any one of claims 14-20, wherein said FVIII variant comprises amino acids 1-740 and 1649-2332 of SEQ ID NO: 1.

22. The FVIII variant of any one of claims 14-21, wherein said FVIII variant comprises amino acids 1-740 and 1690-2332 of SEQ ID NO: 1.

23. A composition comprising at least one FVIII variant of any one of claims 14-22 and at least one pharmaceutically acceptable carrier.

24. A method for treatment of a hemostasis related disorder in a patient in need thereof comprising administering a therapeutically effective amount of the FVIII variant of any one of claims 14-22 in a pharmaceutically acceptable carrier to said patient.

25. The method of claim 24, wherein said hemostasis related disorder is hemophilia.

26. An isolated nucleic acid molecule encoding the FVIII variant of any one of claims 14-22.

27. The nucleic acid molecule of claim 26, wherein said FVIII variant comprises a signal peptide.

28. An expression vector comprising the nucleic acid molecule of claim 26 operably linked to a regulatory sequence.

29. The vector of claim 28, selected from the group consisting of an adenoviral vector, an adenovirus-associated vector, a retroviral vector, a plasmid, and a lentiviral vector.

30. A host cell comprising the vector of claim 29.

31. The host cell of claim 30, wherein said host cells are human cells.

32. A method for treatment of a hemostasis related disorder in a patient in need thereof comprising administering a therapeutically effective amount of the vector of claim 28 in a pharmaceutically acceptable carrier.

33. The method of claim 32, wherein said hemostasis related disorder is hemophilia.

34. The activated form of the FVIII variant of any one of claims 14-22.

35. A method for reducing blood loss in a patient in need thereof comprising administering a therapeutically effective amount of the FVIII variant of any one of claims 14-22 or the nucleic acid molecule of claim 26 in a pharmaceutically acceptable carrier to said patient.
